(19) United States
(12) Patent Application Publication
(10) Pub. No.: US 2025/0140357 A1
(43) Pub. Date: May 1, 2025

(54) BLOODWORK BASED DIABETIC TREATMENT PROTOCOL AND AI PLATFORM IMPLEMENTING THE SAME

(71) Applicants: Darren Dubyak, Gibsonia, PA (US); Alexandra Dubyak, Gibsonia, PA (US)

(72) Inventors: Darren Dubyak, Gibsonia, PA (US); Alexandra Dubyak, Gibsonia, PA (US)

(21) Appl. No.: 18/930,859

(22) Filed: Oct. 29, 2024

Related U.S. Application Data
(60) Provisional application No. 63/546,220, filed on Oct. 29, 2023.

(51) Int. Cl. G16H 10/40; G16H 20/10; G16H 20/60

(52) U.S. Cl. CPC G16H 10/40; G16H 20/10; G16H 20/60

(57) ABSTRACT

A blood based diabetes treatment protocol AI platform includes an AI platform database which includes demographics, such as age, gender, height, weight, occupation, ethnicity, social habits, medications and family history, medical symptoms, and specialized bloodwork panel of a patient; Wherein the AI platform is configured for Analyzing bloodwork and demographics to categorize a patient, and for Assigning treatment regimen including retesting, optional supplementation and medication and for Assigning at least one phase of a three phase diet regimen; and Wherein the AI platform is configured for prediction of the patient weight loss and lowering of Type 2 diabetes and prediabetes markers within a set time period on the protocol.

The present application claims the benefit of U.S. Provisional Patent Application Ser. No. 63/546,220 filed Oct. 29, 2023 titled “Bloodwork Based Diabetic Treatment Protocol and AI Platform Implementing the Same” which is incorporated herein by reference.

### BACKGROUND OF THE INVENTION

### 1. Field of the Invention

This invention generally relates to diabetic treatment protocols and AI based diabetic platforms.

### 2. Background Information

### Diabetes Background

Diabetes mellitus, often known simply as diabetes, is a group of common endocrine diseases characterized by sustained high blood sugar levels. The Center for Disease control (CDC) states that “diabetes is a chronic (long-lasting) health condition that affects how your body turns food into energy. Your body breaks down most of the food you eat into sugar (glucose) and releases it into your bloodstream. When your blood sugar goes up, it signals your pancreas to release insulin. Insulin acts like a key to let the blood sugar into your body's cells for use as energy. With diabetes, your body doesn't make enough insulin or can't use it as well as it should. When there isn't enough insulin or cells stop responding to insulin, too much blood sugar stays in your bloodstream. Over time, that can cause serious health problems, such as heart disease, vision loss, and kidney disease. There isn't a cure yet for diabetes, but losing weight, eating healthy food, and being active can really help.”

It is estimated in 2023 that more than 37 million US adults have diabetes, and 1 in 5 of them don't know they have it. Diabetes is the eighth leading cause of death in the United States. Diabetes is the No. 1 cause of kidney failure, lower-limb amputations, and adult blindness. In the last 20 years, the number of adults diagnosed with diabetes has more than doubled.

There are three main types of diabetes: type 1, type 2, and gestational diabetes (diabetes while pregnant). Type 1 diabetes is thought to be caused by an autoimmune reaction (the body attacks itself by mistake) and this reaction stops the body from making insulin. Approximately 5-10% of the people who have diabetes have type 1. Type 1 diabetes can be diagnosed at any age, and symptoms often develop quickly. Currently, no one knows how to prevent type 1 diabetes. With type 2 diabetes, one's body doesn't use insulin well and can't keep blood sugar at normal levels. About 90-95% of people with diabetes have type 2. It develops over many years and is usually diagnosed in adults (but more and more in children, teens, and young adults). One may not notice any symptoms so blood sugar testing is recommended for those at risk. Type 2 diabetes can be prevented or delayed with healthy lifestyle changes, including weight loss, eating healthy and being active. Gestational diabetes develops in pregnant women who have never had diabetes. Gestational diabetes usually goes away after pregnancy, however, it increases ones risk for type 2 diabetes later in life. Further the babies of women suffering gestational diabetes have an increased propensity toward obesity as a child or teen and develop type 2 diabetes later in life.

Additionally relevant is a discussion of prediabetes. In the United States, 96 million adults—more than 1 in 3—have prediabetes. More than 8 in 10 of them don't know they have it. With prediabetes, blood sugar levels are higher than normal, but not high enough for a type 2 diabetes diagnosis. Prediabetes raises the patient's risk for type 2 diabetes, heart disease, and stroke.

### Diabetes Diagnoses

Conventionally diabetes is currently diagnosed using blood tests, such as the A1c test, the glycosolated hemoglobin or hemoglobin A1c test, the oral glucose tolerance test, or the random plasma glucose test. The A1c test and the glycosolated hemoglobin or hemoglobin A1c test are similar and measure the average blood glucose level over the past two to three months. The oral glucose tolerance test measures the blood glucose level after fasting and drinking a glucose-containing beverage. The random plasma glucose test measures the blood glucose level at any time without regard to the last meal.

The American Diabetes Association currently recommends using the A1c test to diagnose diabetes and prediabetes and provides the following criteria: A1c levels over 6.5% are diagnostic of diabetes; values between 5.7-6.4% are diagnostic of prediabetes; and test results less than 5.6% are normal.

The diagnosis of diabetes can also be confirmed by the presence of symptoms, such as excessive thirst, frequent urination, weight loss, nausea, abdominal pain, heavy breathing with the smell of acetone in the breath, drowsiness, and diabetes coma, along with elevated blood glucose greater than 11.1 mmol/L.

The medical community's relies heavily upon treating diabetes with the use of pharmaceuticals, many becoming lifelong maintenance medications. However, many individuals seek alternative approaches to managing and reversing their diabetes. The goal of managing and reversing their diabetes without the use of lifelong pharmaceuticals is possible for prediabetes and type 2 diabetes.

### Artificial Intelligence

Artificial intelligence (AI) refers to the creation of computer systems capable of performing tasks that historically only a human could do, such as reasoning, making decisions, or solving problems. AI may be better defined and the theory and development of computer systems capable of performing tasks that historically required human intelligence, such as recognizing speech, making decisions, and identifying patterns. AI is an umbrella term that encompasses a range of technologies, including machine learning, deep learning, and natural language processing (NLP).

Machine learning is a subset of artificial intelligence that aims to create computer systems that discover patterns in training data to perform classification and prediction tasks on new data. Machine learning puts together tools from statistics, data mining, and optimization to generate models.

Representational learning, a subarea of machine learning, focuses on automatically finding an accurate representation of the knowledge extracted from the data. When this representation comprises many layers (i.e., a multi-level representation), we are dealing with deep learning.

In deep learning models, every layer represents a level of learned knowledge. The nearest to the input layer represents low-level details of the data, while the closest to the output layer represents a higher level of discrimination with more abstract concepts.

AI, namely machine learning, has been utilized in in diabetes prediction and diagnosis platforms. For one example see Machine Learning Based Diabetes Classification and Prediction for Healthcare Applications, by Umair Muneer Butt et al, Journal of Healthcare Engineering, Oct. 1, 2021 in which a machine learning based approach has been proposed for the classification, early-stage identification, and prediction of diabetes. This platform is suggested to presents an IoT-based hypothetical diabetes monitoring system for a healthy and affected person to monitor his blood glucose (BG) level. In this platform, for diabetes classification, three different classifiers have been employed, i.e., random forest (RF), multilayer perceptron (MLP), and logistic regression (LR). For predictive analysis, the platform employed long short-term memory (LSTM), moving averages (MA), and linear regression (LR). A benchmark PIMA Indian Diabetes dataset was used in implementing this platform.

There have been a number of such systems such that studies of a collection of such systems have been published. For reference, Chaki et al. (Chaki J, Ganesh S T, Cidham S K, Theertan S A. Machine learning and artificial intelligence based diabetes mellitus detection and self-management: a systematic review. J King Saud Univ Comput Inf Sci. 2020) reviewed machine learning models in diabetes detection. The review included 107 studies and classified them according to the model or classifier, the dataset, the features selection with four possible kinds of features, and their performance. The authors found that text, shape, and texture features produced better outcomes. Also, they found that DNNs and SVMs delivered better classification outcomes, followed by RFs. Finally, Silva et al. (Silva K D, Lee W K, Forbes A, Demmer R T, Barton C, Enticott J. Use and performance of machine learning models for type 2 diabetes prediction in community settings: a systematic review and meta-analysis. Int J Med Inform) reviewed 27 studies, including 40 predictive models for diabetes. They extracted the technique used, the temporality of prediction, the risk of bias, and validation metrics. The objective was to prove whether machine learning exhibited discrimination ability to predict and diagnose type 2 diabetes. Although this ability was confirmed, the authors did not report which machine learning model produced the best results.

There remains a need in the art to better diagnose and treat diabetic conditions and to do so with a patient centered approach.

### SUMMARY OF THE INVENTION

The various embodiments and examples of the present invention as presented herein are understood to be illustrative of the present invention and not restrictive thereof and are non-limiting with respect to the scope of the invention.

The present invention provides a blood based diabetes treatment protocol helping patients achieve improved health goals through personalized treatment plans effectively lowering Type 2 diabetes and prediabetes markers. The protocol incorporates four individualized aspects Custom IV Therapy, Nutraceuticals, Personalized Health Coaching, and Medications which are designed to promote weight loss while effectively lowering Type 2 diabetes and prediabetes markers. An AI predictive platform implementing the protocol can demonstrate advantageous outcomes enhancing effectiveness of the protocol.

It is a fundamental goal of the protocol of the invention to empower individual patients to take control of their health through personalized and science-driven approaches that enhance weight loss and metabolic health plans effectively lowering Type 2 diabetes and prediabetes markers, and minimizing patients that require lifelong medications.

One aspect of the present invention comprises a diabetes treatment protocol comprising the steps of: Obtaining demographics, such as age, gender, height, weight, occupation, ethnicity, social habits, medications and family history, medical symptoms, and specialized bloodwork panel of patients; Analyzing bloodwork and demographics categorize patient into distinct categories such as i) NOT REMARKABLE ii) WATCHLIST iii) PRE-DIABETIC iv) TYPE 2 and v) UNCONTROLLED Type 2; assigning treatment regimen including retesting, optional supplementation and medication; assigning at least one phase of a three phase diet regimen; and assigning a maintenance and monitoring regimen including virtual health coaching, diet tools including food scale, water bottle and measuring cups, and selective glucose monitors; and retesting at designated time period

Another aspect of the present invention comprises an AI diabetes machine learning platform for implementing the diabetes treatment protocol discussed above.

The present invention is based upon the observations of the inventors that Type II diabetes can often be attributed to two factors: knowledge and choices. The food patients consume plays a critical role in their health, and often it is a lack of food knowledge representing a large factor contributing to diabetes. The treatment protocol and AI platform set forth herein aids in preventing and reversing Type II Diabetes. The patients in the treatment protocol of the invention have been demonstrated to respond quicker and faster than any medication to lower their blood glucose and HA1C levels.

These and other advantages of the present invention will be clarified in the description of the preferred embodiments taken together with the attached figures.

### BRIEF DESCRIPTION OF THE FIGURES

FIG. 1 is a schematic view of a patent centric bloodwork based diabetic treatment platform according to the present invention.

FIG. 2 is a schematic flow chart of the patent centric bloodwork based diabetic treatment protocol for a patient using the platform of FIG. 1.

FIG. 3 is a schematic view of the development of the AI platform implementing a patent centric bloodwork based diabetic treatment protocol used in the platform of FIG. 1.

### DESCRIPTION OF THE PREFERRED EMBODIMENTS

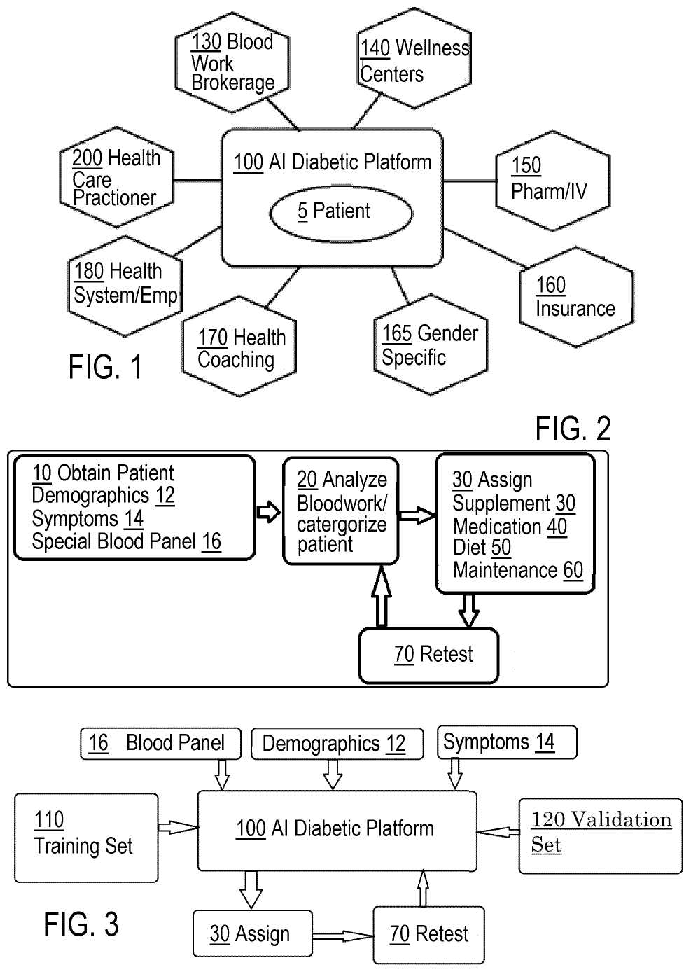

The bloodwork based diabetic treatment protocol according to the present invention begins by focusing upon the individual patient 5. The protocol is implemented on a cloud based AI predictive diabetic platform 100 schematically represented in FIG. 1. The platform 100 as described below will utilize an electronic health record (EHR) system integrated with the AI predictive diabetic platform 100, allowing for seamless tracking of client progress, secure data management, and effective communication between stakeholders. As detailed below a blood based diabetes treatment protocol AI platform 100 according to the invention includes an AI platform database which includes demographics, such as age, gender, height, weight, occupation, ethnicity, social habits, medications and family history, medical symptoms, and specialized bloodwork panel of a patient; Wherein the AI platform is configured for Analyzing bloodwork and demographics to categorize a patient, and for Assigning treatment regimen including retesting, optional supplementation and medication and for Assigning at least one phase of a three phase diet regimen; and Wherein the AI platform is configured for prediction of the patient weight loss and lowering of Type 2 diabetes and prediabetes markers within a set time period on the protocol.

The treatment protocol utilizes a specialized blood panel 16, discussed in detail below, as a definitive tool to assess and customize the treatment protocol for the patient. The symptoms and demographics of the patient 5 are also utilized to create a customized treatment protocol as detailed below. The comprehensive protocol is designed to effectively and efficiently improve the health and wellness of the patient 5. The comprehensive protocol can implement nutraceuticals (as needed) medication (as needed), health coaching and meal planning with retesting to reevaluate the treatment.

The treatment protocol of the invention uses a baseline analysis 10 of a patient including Demographic information 12, Patient Diabetic Symptoms 14 and Bloodwork 16 to diagnoses and treat diabetes in patients.

### Step 1

The first step of the treatment protocol of the present invention is to obtain demographics 12 (age, gender, height, weight, occupation, ethnicity, social habits, medications and family history) of a patient 5, medical symptoms 14 (diabetes symptoms) of a patient 5, and specialized bloodwork panel 16 of a patient 5, wherein the specialized blood panel 16 including these variables fasting glucose hemoglobin, A1C, CMP, Iron Studies, Vitamin b12, Folate, Vitamin D, Lipid Panel, Creative Protein CRP, TSH, Free T3, Free T4, TPO antibody, Thyroglobulin Antibody, Cortisol, Testosterone test (free and total) Progesterone, Estradiol, Insulin, and CBC.

### Demographic Information 12

The demographic information 12 of the patient 5 includes the weight of the patient 5 together with the height of the patient 5 and biological gender of the patient 5. The height and weight of a patient 5 have proven to be important factors in evaluating risks and progression of diabetes. An estimated 80% to 90% of people with type 2 diabetes are categorized as overweight or obese. The CDC notes that patients with too much belly fat increase their risk for type 2 diabetes. There is debate on proper measurements for obesity and overweight classification, however despite these objections or limitations these factors are certainly indicative of a “healthy weight” for the patient and are highly relevant for the diagnosis and treatment of diabetes.

The gender of the patient 5 is significant for the protocol on the platform 100. FIG. 1 is a schematic view of the patent centric bloodwork based diabetic treatment platform 100 generally showing the interaction of separate stakeholders through separate portals (130 bloodwork brokerage houses; 140 wellness centers, 150 Pharmaceutical and IV treatment providers, 160 insurance providers, 170 health coaches, 180 health system or employer, or 200 health care practitioner or physician) to the cloud based platform 100 according to the present invention. Another aspect or interaction of the platform is a gender specific “filter” 165. The gender divisions are significant in the platform 100, and males are achieving significantly improved results than females in early testing of the protocol used on the platform 100. The difference is so stark that this is shown as a different portal for the platform 100 because the results are so dramatically different.

The demographic information 12 includes the family history of the patient, relevant to diabetes. Family history has been established to be a strong, independent and easily assessed risk factor for type 2 diabetes. This elevated risk of type 2 diabetes based upon a family history is attributed, in part, to both genetic and shared environmental components amongst family members, but the precise factors accounting for this increase in risk are poorly understood and subject to some debate. Family history remains a relevant and obtainable factor.

The demographic information 12 includes the age of the patient 5. Diabetes is more prevalent in older populations and older populations are more likely to have experienced sever adverse effects of diabetes. According to the Centers for Disease Control and Prevention, 50 percent of adults age 65 and older have prediabetes and 25 percent have diabetes.

The demographic information 12 includes the ethnicity of the patient 5. Ethnicity of patients 5 have proven to be relevant in evaluating diabetes risks and prognosis. For example, people of certain racial and ethnic groups are more likely to develop type 2 diabetes. These groups include people of Asian, African, and Afro-Caribbean ethnicity. Ethnicity of a patient 5 remains highly relevant for the diagnosis and treatment of diabetes.

The demographic information 12 includes the social habits or lifestyle (also called environmental factors) of the patient 5. Environmental and lifestyle factors, in addition to the ageing of populations, are generally believed to account for the rapid global increase in type 2 diabetes prevalence and incidence in recent decades. These lifestyle factors include level of physical activity (exercise), increased monitor viewing time or sitting in general (often occupation related), exposure to noise or fine dust, short or disturbed sleep, smoking, stress and depression (and stress management), alcohol consumption, illness, menstruation, menopause and socioeconomic status. For reference, many of these factors can promote body changes inducing the development of overt type 2 diabetes. These environmental and lifestyle factors are relevant in diabetes prediction for the patient 5 and treatment and experts have noted that multiple mechanistic pathways may come into play.

The demographic information 12 includes the occupation of the patient 5, generally related to sedation and stress levels of the patient 5, although some professions may raise sleep issues as well.

The demographic information 12 represents a multitude of factors or variables that allow the AI platform 100 implementing the Bloodwork Based Diabetic Treatment Protocol of the present invention to better modify and improve the protocol and to predict the expected lowering Type 2 diabetes and prediabetes markers for a given patient 5 following the protocol.

The AI platform 100 is well suited for independently tracking this large number of variables represented by the demographic information 12 and identifying patterns of improved results or less favorable results within the variables. The AI platform 100 is intended to utilize the demographic information 12 to customize the treatment protocol for the patients 5 and, separately to predict the expected lowering Type 2 diabetes and prediabetes markers for a given patient 5 following the protocol. The second aspect, the predictive model in the platform 100 is to give the patient 5 (and other relevant stakeholders 140, 200, etc.) an additional tool and incentive to make informed decisions and better choices.

### Medical Symptoms 14 (Diabetes Symptoms)

The protocol implemented in the platform 100 obtains the medical symptoms 14 (diabetes symptoms) of the patient 5, which is often why certain patients 5 are coming for treatment to their health care practitioner 200 or wellness center 140. Diabetes symptoms for a given patient 5 generally depend on the blood sugar level of the patient 5. Some patients 5, especially if they have prediabetes, gestational diabetes or type 2 diabetes, may not have symptoms. In type 1 diabetes, symptoms tend to come on quickly and be more severe. Some of the symptoms of type 1 diabetes and type 2 diabetes patients 5 are: Feeling more thirsty than usual; Urinating often; Losing weight without trying; Presence of ketones in the urine (ketones are a byproduct of the breakdown of muscle and fat that happens when there's not enough available insulin; Feeling tired and weak; Feeling irritable or having other mood changes; Having blurry vision; Feeling very hungry; Having numb or tingling hands or feet; having very dry skin; Having slow-healing sores; and Getting a lot of infections, such as gum, skin and vaginal infections.

The medical symptoms 14, like the demographic information 12 represents a multitude of factors of variables that allow the AI platform 100 implementing the Bloodwork Based Diabetic Treatment Protocol of the present invention to better modify and improve the protocol. The AI platform 100 is essentially exceptionally well suited for independently tracking this large number of variables represented by the medical symptoms 14, like the demographic information 12, and identifying patterns of improved results or less favorable results within the variables. The AI platform 100 is intended to utilize the symptoms 14 to customize the treatment protocol for the patients 5. The second, and equally important aspect of the AI platform is to form a predictive model of the Type 2 diabetes and prediabetes markers for a given patient 5 following the proposed protocol assigned by the platform 100. Patients 5 given a physical target or expected result can motivate patients 5 to invest in (monetarily, physically and mentally) engage with and follow the proposed protocol with the ultimate goal of reducing the number of patients 5 on life-long medication.

### Specialized Bloodwork Panel 16

The protocol implemented in the platform 100 obtains a specialized bloodwork panel 16 of patients 5. The protocol of the present invention is bloodwork based and this is at the heart of the invention. The specialized blood panel including these variables patient A1C, Fasting blood glucose (FBG), Comprehensive Metabolic Panel (CMP), Iron status blood tests, Vitamin B12 (cobalamin), Folate, Vitamin D, Lipid Panel, C-reactive protein (CRP), TSH, T3 test (primarily Free T3), T4 test (Primarily Free T4), TPO antibody test, Thyroglobulin Antibody, Cortisol, Testosterone test (free and total), Progesterone, Estradiol (E2), Insulin, CBC.

A1C, or A1C test, measures what percentage of hemoglobin proteins in the blood of a patient 5 which are coated with sugar (glycated). Hemoglobin proteins in red blood cells transport oxygen. The A1C test is also called the glycated hemoglobin, glycosylated hemoglobin, hemoglobin A1C or HbA1c test. This testing was originally developed at least as early as the 1960s and its use as marker of glycemic control has gradually increased over the course of the last four decades. Recognized as the gold standard of diabetic survey, this parameter was successfully implemented in clinical practice in the 1970s and 1980s and internationally standardized in the 1990s and 2000s. The use of standardized and well-controlled methods, with well-defined performance criteria makes A1C testing optimal for use in care of the patient 5, e.g., for diabetes diagnosis. Many reports devoted to HbA1c, or simply A1C, have been published in numerous peer reviewed journals.

Fasting blood glucose (FBG) is a blood test that measures the amount of glucose in the blood of a patient 5 after the patient 5 has fasted for at least eight hours. Fasting blood glucose (FBG) and glycosylated hemoglobin (HbA1c) are recognized as key indicators reflecting blood glucose control in type 2 diabetes mellitus (T2DM) patients 5. For further background on this variable and also on methods and state of the art of developing machine learning platforms in the diabetic field, please see Tao, X., Jiang, M., Liu, Y. et al. Predicting three-month fasting blood glucose and glycated hemoglobin changes in patients with type 2 diabetes mellitus based on multiple machine learning algorithms. Sci Rep 13, 16437 (2023). This paper discusses the creation of a predictive AI diabetic predictive model for blood glucose changes in type 2 diabetes patients after 3 months of treatment. These machine learning algorithms are analogous to that implemented in the platform 100. Other AI algorithms may be used, for example H2O.AI is a company that offers an open source enterprise framework products for machine learning and the platform 100 may be easily implemented on this type of framework which will generate predictive algorithms based upon a review of positive correlations between identified datasets. It should be understood the specific algorithms and weights of variable therein will continuously update and modify as more data is added in the machine learning process. Microsoft has an AZURE platform that may be equally applicable for the platform 100.

A comprehensive metabolic panel (CMP) is a blood sample test that measures 14 different substances in a patient's blood. It provides important information about the patient's chemical balance and metabolism (how the body transforms the food one eats into energy). A CMP measures: most significantly glucose; Calcium, which is one of the most important and common minerals in the body of the patient 5. While most of a body's calcium is stored in bones, one needs calcium in blood as well, as Blood calcium is essential for proper functioning of the nerves, muscles and heart of a patient 5; total protein which is a measurement of the total amount of albumin and globulins, which are proteins in the blood; Bilirubin namely a waste product that's made from the breakdown of red blood cells and the liver is in charge of removing bilirubin from the body; blood urea nitrogen (BUN) which is a measurement of urea nitrogen, which is a waste product that the kidneys help remove from the blood; Creatinine, which is a byproduct of muscle activity (a waste product that the kidneys filter and remove from the blood; albumin, a protein that the liver makes which transports important substances through the bloodstream and keeps fluid from leaking out of the blood vessels; Sodium, which mostly comes from the food one eats, and the kidneys help regulate the body's sodium levels; Potassium, which also primarily comes from the food one eats and is present in all tissues of the body; Bicarbonate which indicates the amount of carbon dioxide (CO₂) in the blood; Chloride functions along with sodium, potassium and bicarbonate to control many body processes; the liver enzyme alkaline phosphonate (ALP); the liver enzyme alanine transaminase (ALT); and the liver enzyme aspartate Aminotransferase (AST).

Iron status blood test is a series of iron blood tests. It is also known as Iron blood panel test, Iron indices blood test. Iron is a mineral in the body that comes from foods like red meat and fortified cereals or from supplements. The body needs iron to make red blood cells. Iron is also an important part of hemoglobin, a protein in the blood that helps carry oxygen from the lungs to the rest of the body. There are several different tests forming the iron studies that show how much of the mineral is moving through the blood, how well the blood carries it, and how much iron is stored in the tissues. These include a Serum iron test which measures the amount of iron in the blood; a serum ferritin test which measures how much iron is stored in the body (When iron level is low, the body will pull iron out of “storage” to use); a Total iron-binding capacity (TIBC) test which measures or identifies how much transferrin (a protein) is free to carry iron through the blood (If the TIBC level is high, it means more transferrin is free because the patient has low iron); a Unsaturated iron-binding capacity (UIBC) test that measures how much transferrin isn't attached to iron; and a Transferrin saturation test which measures the percentage of transferrin that is attached to iron.

Vitamin B12 (cobalamin) is a water-soluble vitamin that is derived from animal products such as red meat, dairy, and eggs. Intrinsic factor is a glycoprotein that is produced by parietal cells in the stomach and necessary for the absorption of B12 in the terminal ileum. Vitamin B-12 is a critical vitamin for many bodily functions, such as brain health, blood cell production, and proper nerve functioning.

Folate is an important nutrient for making red blood cells and for repairing cells and nerve tissue in the body. The folate blood test examines levels of folate in the blood (serum folate) and red blood cells (RBC folate).

Vitamin D is technically not a vitamin but rather can be considered to be a hormone, and is a fat-soluble vitamin that plays a vital role in calcium absorption to give the patient 5 healthy and strong bones. Vitamin D is a group of fat-soluble secosteroids It exists in two forms: Vitamin D2 (ergocalciferol) obtained from rom vitamin D-fortified foods such as cereals, milk and yogurt; and Vitamin D3 (cholecalciferol) in which the body makes vitamin D3 when it gets exposed to sunlight. It is also found in foods such as eggs and fish, such as salmon, sardines, tuna and mackerel. Both the forms, vitamin D2 and vitamin D3, get converted to another form of vitamin D called 25 hydroxyvitamin D or 25(OH)D in the body. The blood test that gives the blood levels of 25(OH)D.

A lipid panel blood test measures the amount of certain fat molecules called lipids in the blood. The panel includes four different cholesterol measurements and a measurement of triglycerides. Here are some of the key measurements that are included in a lipid panel: Total cholesterol which is an overall cholesterol level-the combination of LDL-C, VLDL-C and HDL-C; Low-density lipoprotein (LDL) cholesterol which is the type of cholesterol that's known as “bad cholesterol” (It can collect in blood vessels and increase the risk of cardiovascular disease); Very low-density lipoprotein (VLDL) cholesterol which is a type of cholesterol that's usually present in very low amounts when the blood sample is a fasting samples since it's mostly comes from food recently eaten (An increase in this type of cholesterol in a fasting sample may be a sign of abnormal lipid metabolism); High-density lipoprotein (HDL) cholesterol which is the type of cholesterol that's known as “good cholesterol” (It helps decrease the buildup of LDL in the blood vessels); and Triglycerides which is a type of fat from the food (Excess amounts of triglycerides in the blood are associated with cardiovascular disease and pancreatic inflammation).

A C-reactive protein (CRP) test is a blood test used to detect general inflammation. Inflammation is the body's natural response to infection, disease, or injury, so a CRP test can provide the first clue as to whether some sort of inflammatory condition is occurring.

The blood panel 16 includes a series of thyroid tests. A TSH test measures the amount of thyroid stimulating hormone (TSH) in the blood of the patient 5. TSH is produced by the pituitary gland. It prompts the thyroid gland to make and release thyroid hormones into the blood. Triiodothyronine (T3) is a thyroid hormone. It plays an important role in the body's control of metabolism (the many processes that control the rate of activity in cells and tissues). T4 (thyroxine) is the main hormone produced by the thyroid gland. Thyroid peroxidase (TPO) is an enzyme made in the thyroid gland that is important in the production of thyroid hormone. TPO is found in thyroid follicle cells where it converts the thyroid hormone T4 to T3. The TPO antibody test, also known as the thyroid peroxidase test, is a test that measures the level of an antibody that is directed against thyroid peroxidase (TPO). Autoantibodies to thyroid peroxidase (TPOAb) are produced within the body. The presence of TPOAb in the blood reflects a prior attack on the thyroid tissue by the body's immune system. A thyroglobulin antibody (TgAb) test looks for certain antibodies that attack the thyroid. The presence of TbAb may indicate an autoimmune condition.

The cortisol blood test measures the level of cortisol in the blood. Cortisol is a steroid (glucocorticoid or corticosteroid) hormone produced by the adrenal gland that helps regulate metabolism, immune response and stress level.

A testosterone test measures the body's levels of the hormone testosterone. Testosterone is the main sex hormone in men, but women also have testosterone in their bodies. Both high and low levels can be indicative of health concerns. Testosterone in the body exists as free testosterone (not attached to anything) and bound testosterone (attached to proteins). Free testosterone is easier for the body to use. The testosterone blood test shows total testosterone and free testosterone levels.

The progesterone blood test measures the level of progesterone the blood. Progesterone is an endogenous steroid and progestogen sex hormone involved in the menstrual cycle, pregnancy, and embryogenesis

An estradiol (E2) test measures levels of the hormone E2 in a person's blood. E2 is one of the four types of estrogen that the ovaries chiefly produce. The adrenal glands, placenta, testes, and some tissues also produce smaller amounts of E2. The results can reflect issues ranging from fertility problems to liver damage.

The insulin blood test is a medical examination that measures the amount of insulin in the bloodstream. It is also known as a fasting insulin test or serum insulin test. The test evaluates insulin resistance, which is frequent in illnesses such as type 2 diabetes and metabolic syndrome. It aids in the diagnosis and management of diabetes-related disorders by assisting healthcare practitioners in understanding how effectively the body responds to insulin.

A complete blood count (CBC) is a blood test used to look at overall health and find a wide range of conditions, including anemia, infection and leukemia. A complete blood count test measures the following: Red blood cells, which carry oxygen; White blood cells, which fight infection; Hemoglobin, the oxygen-carrying protein in red blood cells; Hematocrit, the amount of red blood cells in the blood; Platelets, which help blood to clot. A complete blood count can show unusual increases or decreases in cell counts. Those changes might point to a medical condition that calls for more testing.

### Step 2

The second step of the treatment protocol implemented in the platform 100 of the present invention is to analyze (step 20) demographics 12, medical symptoms 14 (diabetes symptoms), and specialized bloodwork panel 16 of patients and categorize (step 30) the patient 5 into: i) NOT REMARKABLE ii) WATCHLIST iii) PRE-DIABETIC iv) TYPE 2, and v) UNCONTROLLED Type 2. Other types of diabetes, namely type 1 and gestational, are possible but fall outside of this treatment protocol and would be treated separately.

### Not Remarkable

The Not Remarkable category of patients 5 have A1C FBG and insulin within normal limits. Normal limits may be according to CDC guidelines. As an example herein normal would be A1C of 5.6 and below and FBG lower than 100 and Insulin levels lower than 30.

### Watchlist

The Watchlist category of patients 5 have A1C within normal limits but at least one or both FBG and insulin outside normal limits. For example, A1C of 5.6 and below and at least one FBG higher than 100 OR insulin levels higher 30.

### Pre-Diabetic

The Prediabetic category of patients 5 have A1C elevated above normal but below the threshold for diabetes, e.g., A1C level within the range of 5.7-6.4.

### Type 2

The Type 2 category of patients 5 have A1C elevated above prediabetes but below the threshold for Un-controlled diabetes, e.g., A1C level within the range of 6.5-8.4.

### Uncontrolled Type 2

The Uncontrolled Type 2 category of patients 5 have A1C elevated within a conventional type 2 diagnosis range, e.g., an A1C level above 8.4)

### Step 3 Assigning Treatment Regimen 30

The third step of the protocol implemented in the platform 100 of the present invention is assigning 30 a treatment regimen to the patient 5 based upon categorization.

For Not remarkable patients 5, they would be given supervised virtual nutritional health coaching (accessed via portal 170) and retesting 70 of patients 5 recommended for 6 months. The demographic information 12 may warrant adjustment of the retesting period 70 as verified by the AI platform 100.

For Watchlist Patients 5 they would be given supervised virtual nutritional health coaching (accessed via portal 170) and supplementation 30 as designated by specialized panel 16 and monitoring by retesting 70 in 60 days. The demographic information 12 may warrant adjustment of the retesting 70 period as verified by the AI platform 100. Supplementation 30 of patients based upon the blood panel 16 is a significant part of the present protocol. The supplementation 30 in the present protocol is typically through dietary supplements 30 which may also be defined as nutraceuticals herein (although the term nutraceutical has no official legal definition within the U.S). As the supplements 30 are not medication 40 they need not be supplied by pharmacological suppliers (represented via portal 150). Wellness centers 140 or practitioner 200, health system 180 may actually source the supplements 30.

In the United States, the Dietary Supplement Health and Education Act (DSHEA) of 1994 defined the term “dietary supplement”: “A dietary supplement 30 is a product taken by mouth that contains a ‘dietary ingredient’ intended to supplement the diet. The ‘dietary ingredients’ in these products may include: vitamins, minerals, herbs or other botanicals, amino acids, and substances such as enzymes, organ tissues, glandulars, and metabolites. Dietary supplements can also be extracts or concentrates, and may be found in many forms such as tablets, capsules, softgels, gelcaps, liquids, or powders.” Dietary supplements 30 do not have to be approved by the U.S. Food and Drug Administration (FDA) before marketing, but companies must register their manufacturing facilities with the FDA and follow current good manufacturing practices (cGMPs). With a few well-defined exceptions, dietary supplements 30 may only be marketed to support the structure or function of the body, and may not claim to treat a disease or condition, and must include a label that says: “These statements have not been evaluated by the Food and Drug Administration. This product is not intended to diagnose, treat, cure, or prevent any disease.” The exceptions are when the FDA has reviewed and approved a health claim. In those situations the FDA also stipulates the exact wording allowed.

The supplements 30 are advised for patients 5 based upon deficiencies or irregularities in the blood panel 16 and are associated with known supplements 30 in known amounts for treating such irregularities. Additionally an important aspect of the AI platform 100 implementation of the protocol is identifying “successful supplements” (or “supplements that retard the progress” of patients) and increasing the number of patients 5 (e.g., similarly situated patients) that should be assigned the “successful supplements” and/or removed from the supplements that retard progress. This is largely the tracking of similarly situated patients 5 and evaluating which supplements 30 provide a statistically significant improved result or identify which supplements 30 provide a statistically significant worse result. The AI platform 100 allows for performing this evaluation over a substantially large number of variables.

For pre-diabetic patient 5, the patient 5 is assigned supervised virtual nutritional health coaching (via portal 170), supplementation 30 as designated by specialized panel, selective medication 40, including selective customized IV therapy (accessed or provided via portal 150) and monitoring 60 followed by retesting 70 in 60 days.

The medication 40 for such pre-diabetic patient 5 may be one of i) Opioid receptor blocking medication and ii) metformin synergy. Metformin synergy is a stack of three medications/nutrients that synergistically stimulate Sirt1, an enzyme that regulates energy and fat metabolism, and B12, a vitamin that boosts energy levels, reduces fatigue, and kick-starts an otherwise sluggish metabolism.

Further including selective customized nutritional IV therapy based upon bloodwork panel 16. IV nutritional therapy is a type of therapy that delivers vitamins, minerals and other nutrients to the body through an IV line inserted into a vein. It generally can also bypass the digestive system and ensure higher absorption of nutrients than oral supplements. The therapy is customized to suit the unique needs of each patient 5. The duration of the infusion depends on the nutrients used and can typically range from 5 minutes to 45 minutes

For Type 2 diabetic patient 5, the patient 5 is assigned supervised virtual nutritional health coaching (via 170 portal), supplementation 30 as designated by specialized panel, medication, selective customized pharmaceutical and/or IV therapy 40 (supplied by providers via portal 150) and monitoring 60 followed by retesting 70 in 60 days. The medication 40 for such type 2 diabetic patient 5 may be one of i) Opioid receptor blocking medication and ii) metformin synergy.

For Uncontrolled Type 2 diabetic patient 5, the patient 5 is assigned supervised virtual nutritional health coaching (via 170 portal), supplementation 30 as designated by specialized panel 16, medication and or selective customized IV therapy 40 and monitoring 60 followed by retesting 70 in 60 days. The medication for such uncontrolled type 2 diabetic patient is one of i) Opioid receptor blocking medication, ii) GLP-1 receptor medication and iii) metformin synergy.

### Step 4 Assign Diet Regimen

The fourth step of the protocol implemented in the platform 100 of the present invention is assigning a diet regimen 50 to the patient

The protocol is based upon a base three phase diet program 50. Phase 1 is essentially an 800-1000 caloric intake/day omitting grains and dairy for duration 30 days with caloric intake of less than 30% fat per day. Phase 2 is essentially an 1100-1600 caloric intake/day with reintroducing of high fiber, low glycemic (less than 100) index food and healthy fats and complex carbohydrates for duration of 30 days. Phase III is essentially a 1200-2000 caloric intake/day with the same food mix of phase II.

The protocol of the present invention will assign pre-diabetic patients, type 2 diabetic patients 5 and uncontrolled diabetic patients 5 at Phase 1 with individual adjusting caloric intake for patient weight and other demographics. The protocol of the present invention will start not remarkable and watchlist patients 5 at phase I or phase II depending upon patient weight.

### Step 5 Maintenance and Monitoring Regimen 60

A key aspect of the present invention is giving patients the tools needed to succeed. Maintenance and monitoring of the patients will assist in this endeavor. Virtual health coaching via portal 170 is provided to all patients and is a form of maintenance and monitoring.

Additionally all patients 5 except the not remarkable are provided with a food scale, water bottle and measuring cups. A simple food scale assists patients 5 in measuring proper portions for their diet and has proven to be one of the greatest aids for people sticking on a portion control diet. Drinking water is regularly cited as a key component of a more healthful diet and patients 5 are recommended to carry a water bottle and fill it throughout the day. Providing a water bottle reinforces this critical component. Likewise a set of measuring cups is extremely helpful for portion control for dieting 50.

The protocol may also assign glucose monitors to pre-diabetic, Type 2 and Uncontrolled type 2 patients 5, and may automatically collect data (e.g., measurement is uploaded automatically)

### Step 6

Another step of the present protocol is retesting 70 and adjust patient treatment accordingly. Retesting is typically at 60 or 90 day increments until significant improvement and/or patient stabilization is obtained.

### AI Platform 100

FIGS. 1-2 are schematic views of the AI platform 100 implementing the Bloodwork Based Diabetic Treatment Protocol according to invention described above. FIG. 1 is a schematic view of the patent centric bloodwork based diabetic treatment platform 100 generally showing the interaction of separate stakeholders through separate portals to the cloud based platform 100 according to the present invention. Reference to the stakeholders and their individualized portals that access the platform 100 are used interchangeably herein.

One stakeholder is the 130 bloodwork brokerage houses who will interact with the platform 100 generally at the direction of the healthcare practitioners or physicians 200 ordering the panel 16. The brokerage houses 130 will be adding the patient 5 panels 16 results directly into the database of the platform 100 and such results will be accessible to the physician 200 together with a proposed personalized plan and a prediction of results for the patient 5 to assist the patient 5 in making informed decision on their own care. A wellness center 140 may have physicians 200 on staff such that they are working with the brokerage houses and the portals 200 and 140 may be effectively merged in that case. Similarly a health system or employer may have in house physician ordering panels 16 from labs (130) such that the portal 180 and 200 are merged.

One stakeholder with its own portal is the 140 wellness center who will interact with the platform 100 generally to implement the patient specific plan and coordinate with health coaching 170, and pharmaceutical/IV suppliers (150) for medicaments 40. It is envisioned that the health coaches will be employed by or controlled by the wellness centers 140. The wellness centers may be updating the results of the patient at retesting in coordination with healthcare practitioners 200.

One stakeholder with its own portal is the 150 Pharmaceutical and IV treatment providers who supply the medications and IVs 40. These will coordinate with patient physicians 200 and will update the platform 200 on the supplied medications (and including timing and dosage information).

One stakeholder with its own portal is the 160 insurance providers. It is noted that a goal of the present platform 100 is minimizing the number of patients 5 on lifetime maintenance drugs. These aspects will be of significant importance to insurance companies 100 and their portal will allow them access to de-identified data to better coordinate member benefits to take full advantage of the system to presumably improve member health and decrease insurance costs.

One stakeholder with its own portal is the 170 health coaches, who will interact with the patients 5 on the platform 100 following a given plan for the patient 5. As noted above it is anticipated that the health coaches 170 will be controlled and managed by the wellness centers 140, although the health care practitioners 200, health systems 180 could also manage these stakeholders.

One stakeholder with its own portal is the 180 health system or employer. This stakeholder is simply different from the individual practicing physician 200 as it represents a number of members or patients across the health system and typically across a number of physicians 200. This stakeholder could be an employer, a union or a trade association offering the advantages of the platform 100 to employees or members. Providing a distinct managing portal for such stakeholders can maximize the implementation and use of the system and allows such stakeholders to independently peruse de-identified data of a collection of patients 5, like insurance companies 160 to verify the advantage of the system 100.

The most important stakeholder of the invention is the patient 5 and is shown at the center of the platform 100. The patient 5 can interact with all stakeholders of the platform 100 and the significance of the platform is to improve results for patients 5.

The implementation of the protocol with the AI platform 100 as an optimization platform implements existing and known AI technologies to enhance the results for patient 5. The data-processing and predictive capabilities of the AI platform enables all stakeholders to better manage their resources and take a more proactive approach to a particular patients plan. With these technologies, the platform 100 will continuously make more accurate and effective treatment plans and patients 5 can receive more personalized treatments. Artificial intelligence is highly efficient at processing huge volumes of data quickly. It is also able to keep track of changes and adapt according to insights gained. The platform 100 may be considered an AI optimization platform and a predictive platform. AI-driven optimization is incredibly powerful in that it can test thousands of ideas and combinations of ideas and find the top performers within a short space of time. As outlined above the AI is intended to identify successful supplements and those that retard success in similarly situated patients using the demographics and the symptoms and the blood panels.

In order to start the platform 100 a training set 110 and validation set 120 will be used as known in the art. The training set 110 is essentially a ground truth known set of patients 5 that move through the protocol and the AI platform 100 can “learn” the rules, although here the classification rules are simple. The validation set is used to determine if the AI platform 100 is properly functioning. The construction of training sets 120 and validations sets 130 for the AI platform 100 are generally known in the art with the unique feature here being the various data inputs being tracked within the protocol of the present invention, as well as the diet parameters that are unique to the present platform 100. The important aspect of the AI platform 100 is that with every patient 5 that is retested the platform 100 gains more data and more data points for determining and optimizing the protocol.

AI machine learning medical platforms and AI diabetic platforms in particular are well known, in the abstract, as noted above. The present AI diabetic platform 100 will utilize the training set 120, the validation set 130 and all the patients 5 that actually move through the program to develop and improve its predictive model for patients 5. It will initially predict and estimate the prognosis of a given patient 5 under the current protocol based upon the training set 120 and the validation set 130 and patients to date, then will adjust the protocol by adding or subtracting supplements 30 based upon similarly situated patients 5 in the past to see if improved results are predicted. The added supplements 30 are those that yield statistically beneficial results (the “successful supplements”) and the supplements 30 that are reduced or deleted are those supplements 30 that retard the progress of patients in a statistically significant manner. The supplements 30 and medicaments 40 assigned by the protocol will slowly change and be optimized over time to improve results of the program implemented on the platform 100.

Other factors are likewise optimized by the AI platform 100 over time, such as the diet 50 parameters including which phase and what caloric restriction are assigned. The AI platform 100 also may optimize the medications. However, with medications 30 it is more likely that adjustments in dosage within the accepted range would proposed by the AI platform 100 as well as identifying any potentially significant contra-indications that should be reviewed by the health care practitioners (e.g., physicians) 200. The AI platform 100 will merely make suggestions and note observations for the medications 40, as opposed to actually making gradual changes to the assigned supplements 30.

The second component of the AI platform 100 is the predictive AI platform. Following the steps above of: 1. Obtaining demographics, such as age, gender, height, weight, occupation, ethnicity, social habits, medications and family history, medical symptoms, and specialized bloodwork panel of patients; 2. Analyzing bloodwork and demographics categorize patient into i) NOT REMARKABLE ii) WATCHLIST iii) PRE-DIABETIC iv) TYPE 2 and v) UNCONTROLLED Type 2; 3. assigning treatment regimen including retesting, optional supplementation and medication; 4 Assigning at least one phase of a three phase diet regimen; and Assign Maintenance and monitoring regimen including virtual health coaching, diet tools including food scale, water bottle and measuring cups, and selective glucose monitors, the AI platform develops 100 predictions of the patients predicted relevant diabetic numbers and weight if this patient follows the proposed protocol. The prediction is based upon past history of patients assuming average compliance. The prediction will have the estimated standard deviation for 50% of the patients and the top 25% of the patients. The predictive AI model is mainly a personalized incentive tool to give the patient 5 informed personalized projection of potential success. It is helpful to give patients 5 facing these issues concrete positive and personalized materials to assist them in adopting and sticking to the proposed treatment plan.

As described above the platform 100 is configured to empower individuals 5 to take control of their health through personalized and science-driven approaches that enhance weight loss and metabolic health. The protocol implemented on the platform 100 weaves together Custom IV Therapy, Nutraceuticals, Personalized Health Coaching, and Medications designed to promote weight loss while effectively lowering Type 2 diabetes and prediabetes markers. Utilizing the AI predictive platform, the platform 100 promotes superior patient outcomes enhancing our effectiveness. The platform 100 offers an integrated health solutions integrating technology and personalized care that transforms lives. Some of the advantages include the following: The Custom IV Therapy represents tailored intravenous nutrient infusions that optimize hydration, boost energy, and address nutrient deficiencies crucial for weight loss and metabolic health. The Nutraceuticals represents a curated selection of high-quality dietary supplements designed to complement our treatment programs, supporting overall health and effective weight management. The Personalized Health Coaching provides One-on-one coaching sessions focusing on sustainable lifestyle changes, including diet, exercise, and behavior modification, personalized to each client's goals and challenges. The Medication Management offers Prescription medications that facilitate weight loss and improve blood sugar control more effectively than conventional methods, guided by comprehensive blood work analysis. The AI Predictive Platform leverages extensive datasets from previous clients 5 to analyze individual health data, enabling the platform 100 to predict treatment efficacy. This personalized approach enhances the ability of the platform 100 to help clients 5 lower blood sugar levels, promote weight loss, and improve overall quality of life, increasing the likelihood of achieving their health goals, including avoiding lifetime maintenance medications.

While this invention has been particularly shown and described with references to the preferred embodiments thereof, it will be understood by those skilled in the art that various changes in form and details may be made therein without departing from the scope of the invention.

What is claimed is:

1. A diabetes treatment protocol comprising the steps of:
Obtaining demographics, such as age, gender, height, weight, occupation, ethnicity, social habits, medications and family history, medical symptoms, and specialized bloodwork panel of a patient;
Analyzing bloodwork and demographics to categorize a patient;
Assigning treatment regimen including retesting, optional supplementation and medication;
Assigning at least one phase of a three phase diet regimen;
Assigning Maintenance and monitoring regimen including virtual health coaching, diet tools including food scale, water bottle and measuring cups, and selective glucose monitors; and
Retesting at designated time period.

2. The diabetes treatment protocol according to claim 1 wherein the Analyzing bloodwork and demographics to categorize a patient is configured to categorize the patient at least into the following groups: i) NOT REMARKABLE ii) WATCHLIST iii) PRE-DIABETIC iv) TYPE 2 and v) UNCONTROLLED Type 2.

3. The diabetes treatment protocol according to claim 1 wherein the specialized bloodwork panel of the patient includes at least five of these variables: patient A1C, Fasting blood glucose (FBG), Comprehensive Metabolic Panel (CMP), Iron status blood tests, Vitamin B12 (cobalamin), Folate, Vitamin D, Lipid Panel, C-reactive protein (CRP), TSH, T3 test (primarily Free T3), T4 test (Primarily Free T4), TPO antibody test, Thyroglobulin Antibody, Cortisol, Testosterone test (free and total), Progesterone, Estradiol (E2), Insulin, and CBC.

4. The diabetes treatment protocol according to claim 1 wherein the specialized bloodwork panel of the patient includes at least ten of these variables: patient A1C, Fasting blood glucose (FBG), Comprehensive Metabolic Panel (CMP), Iron status blood tests, Vitamin B12 (cobalamin), Folate, Vitamin D, Lipid Panel, C-reactive protein (CRP), TSH, T3 test (primarily Free T3), T4 test (Primarily Free T4), TPO antibody test, Thyroglobulin Antibody, Cortisol, Testosterone test (free and total), Progesterone, Estradiol (E2), Insulin, and CBC.

5. The diabetes treatment protocol according to claim 1 wherein the specialized bloodwork panel of the patient includes at least fifteen of these variables: patient A1C, Fasting blood glucose (FBG), Comprehensive Metabolic Panel (CMP), Iron status blood tests, Vitamin B12 (cobalamin), Folate, Vitamin D, Lipid Panel, C-reactive protein (CRP), TSH, T3 test (primarily Free T3), T4 test (Primarily Free T4), TPO antibody test, Thyroglobulin Antibody, Cortisol, Testosterone test (free and total), Progesterone, Estradiol (E2), Insulin, and CBC.

6. The diabetes treatment protocol according to claim 1 wherein the medication is one of i) Opioid receptor blocking medication and ii) metformin synergy.

7. The diabetes treatment protocol according to claim 1 wherein Assigning Maintenance and monitoring regimen further includes a prediction of the patient weight loss and lowering of Type 2 diabetes and prediabetes markers within a set time period on the protocol.

8. A blood based diabetes treatment protocol AI platform comprising:
An AI platform database which includes demographics, such as age, gender, height, weight, occupation, ethnicity, social habits, medications and family history, medical symptoms, and specialized bloodwork panel of a patient;
Wherein the AI platform is configured for Analyzing bloodwork and demographics to categorize a patient, and for Assigning treatment regimen including retesting, optional supplementation and medication and for Assigning at least one phase of a three phase diet regimen; and
Wherein the AI platform is configured for prediction of the patient weight loss and lowering of Type 2 diabetes and prediabetes markers within a set time period on the protocol.

9. The blood based diabetes treatment protocol AI platform according to claim 8 wherein the AI platform is configured for Analyzing bloodwork and demographics to categorize a patient is configured to categorize the patient at least into the following groups: i) NOT REMARKABLE ii) WATCHLIST iii) PRE-DIABETIC iv) TYPE 2 and v) UNCONTROLLED Type 2.

10. The blood based diabetes treatment protocol AI platform according to claim 8 wherein the specialized bloodwork panel of the patient includes at least five of these variables: patient A1C, Fasting blood glucose (FBG), Comprehensive Metabolic Panel (CMP), Iron status blood tests, Vitamin B12 (cobalamin), Folate, Vitamin D, Lipid Panel, C-reactive protein (CRP), TSH, T3 test (primarily Free T3), T4 test (Primarily Free T4), TPO antibody test, Thyroglobulin Antibody, Cortisol, Testosterone test (free and total), Progesterone, Estradiol (E2), Insulin, and CBC.

11. The blood based diabetes treatment protocol AI platform according to claim 8 wherein the specialized bloodwork panel of the patient includes at least ten of these variables: patient A1C, Fasting blood glucose (FBG), Comprehensive Metabolic Panel (CMP), Iron status blood tests, Vitamin B12 (cobalamin), Folate, Vitamin D, Lipid Panel, C-reactive protein (CRP), TSH, T3 test (primarily Free T3), T4 test (Primarily Free T4), TPO antibody test, Thyroglobulin Antibody, Cortisol, Testosterone test (free and total), Progesterone, Estradiol (E2), Insulin, and CBC.

12. The blood based diabetes treatment protocol AI platform according to claim 8 wherein the specialized bloodwork panel of the patient includes at least fifteen of these variables: patient A1C, Fasting blood glucose (FBG), Comprehensive Metabolic Panel (CMP), Iron status blood tests, Vitamin B12 (cobalamin), Folate, Vitamin D, Lipid Panel, C-reactive protein (CRP), TSH, T3 test (primarily Free T3), T4 test (Primarily Free T4), TPO antibody test, Thyroglobulin Antibody, Cortisol, Testosterone test (free and total), Progesterone, Estradiol (E2), Insulin, and CBC.

13. The blood based diabetes treatment protocol AI platform according to claim 8 wherein the medication is one i) Opioid receptor blocking medication and ii) metformin synergy.

14. The blood based diabetes treatment protocol AI platform according to claim 8 wherein the medication is metformin synergy.

15. An AI platform database which includes patient demographics and specialized bloodwork panel of a patient, wherein the specialized bloodwork panel of the patient includes at least five of these variables: patient A1C, Fasting blood glucose (FBG), Comprehensive Metabolic Panel (CMP), Iron status blood tests, Vitamin B12 (cobalamin), Folate, Vitamin D, Lipid Panel, C-reactive protein (CRP), TSH, T3 test (primarily Free T3), T4 test (Primarily Free T4), TPO antibody test, Thyroglobulin Antibody, Cortisol, Testosterone test (free and total), Progesterone, Estradiol (E2), Insulin, and CBC;
Wherein the AI platform is configured for Analyzing bloodwork and demographics to categorize a patient, and for Assigning treatment regimen including retesting, optional supplementation and medication and for Assigning at least one phase of a three phase diet regimen; and
Wherein the AI platform is configured for prediction of the patient weight loss and lowering of Type 2 diabetes and prediabetes markers within a set time period on the protocol.

16. The blood based diabetes treatment protocol AI platform according to claim 15 wherein the AI platform is configured for Analyzing bloodwork and demographics to categorize a patient is configured to categorize the patient at least into the following groups: i) NOT REMARKABLE ii) WATCHLIST iii) PRE-DIABETIC iv) TYPE 2 and v) UNCONTROLLED Type 2.

17. The blood based diabetes treatment protocol AI platform according to claim 15 wherein the specialized bloodwork panel of the patient includes at least ten of these variables: patient A1C, Fasting blood glucose (FBG), Comprehensive Metabolic Panel (CMP), Iron status blood tests, Vitamin B12 (cobalamin), Folate, Vitamin D, Lipid Panel, C-reactive protein (CRP), TSH, T3 test (primarily Free T3), T4 test (Primarily Free T4), TPO antibody test, Thyroglobulin Antibody, Cortisol, Testosterone test (free and total), Progesterone, Estradiol (E2), Insulin, and CBC.

18. The blood based diabetes treatment protocol AI platform according to claim 15 wherein the specialized bloodwork panel of the patient includes at least fifteen of these variables: patient A1C, Fasting blood glucose (FBG), Comprehensive Metabolic Panel (CMP), Iron status blood tests, Vitamin B12 (cobalamin), Folate, Vitamin D, Lipid Panel, C-reactive protein (CRP), TSH, T3 test (primarily Free T3), T4 test (Primarily Free T4), TPO antibody test, Thyroglobulin Antibody, Cortisol, Testosterone test (free and total), Progesterone, Estradiol (E2), Insulin, and CBC.

19. The blood based diabetes treatment protocol AI platform according to claim 15 wherein the medication is one i) Opioid receptor blocking medication and ii) metformin synergy.

20. The blood based diabetes treatment protocol AI platform according to claim 15 wherein the medication is an Opioid receptor blocking medication.
